(19) United States
(12) Patent Application Publication
(10) Pub. No.: US 2021/0379373 A1
(43) Pub. Date: Dec. 9, 2021

(54) IMPLANTABLE MEDICAL LEADS AND METHODS FOR IMPLANTING IMPLANTABLE MEDICAL LEADS FOR SACRAL MODULATION THERAPY

(71) Applicant: MEDTRONIC, INC., Minnepolis, MN (US)

(72) Inventors: Juan G. Hincapie, Minneapolis, MN (US); Sarah J. Offutt, Minneapolis, MN (US); Jerel K. Mueller, Minneapolis, MN (US); Robert T. Sandgren, Minneapolis, MN (US); Katie Bittner, Minneapolis, MN (US)

(73) Assignee: MEDTRONIC, INC., Minneapolis, MN (US)

(21) Appl. No.: 17/321,859

(22) Filed: May 17, 2021

Related U.S. Application Data
(60) Provisional application No. 63/035,143, filed on Jun. 5, 2020.

(51) Int. Cl. A61N 1/36; A61N 1/05; A61N 1/372; A61N 1/375

(52) U.S. Cl. CPC A61N 1/36007; A61N 1/3606; A61B 5/062; A61N 1/37211; A61N 1/37516; A61N 1/0558

(57) ABSTRACT

An implantable medical lead for sacral modulation therapy is disclosed. The implantable medical lead includes a lead body having a distal portion. An electrode is electrically coupled to the lead body and configured to generate a stimulation field or to sense electrical fields. A fixation mechanism is coupled to the distal portion. The fixation mechanism is configured to anchor the implantable medical lead against an interior wall of a blood vessel. The electrode can effect the stimulation field from within the vessel to stimulate a selected sacral nerve or sense electrical signals such as muscle or nerve responses. Guided implantation of the medical lead or other medical leads through the body to a nerve of interest for neurostimulation via remote sensor is also disclosed.

### CROSS-REFERENCE TO RELATED APPLICATION

This Non-Provisional Utility Application claims benefit to U.S. Provisional Application No. 63/035,143, filed Jun. 5, 2020, titled “IMPLANTABLE MEDICAL LEADS AND METHODS FOR IMPLANTING IMPLANTABLE MEDICAL LEADS,” the entirety of which incorporated herein by reference.

### BACKGROUND

The present disclosure relates generally to a method and apparatus that allows for electrical stimulation of body tissue, such as sacral nerves for the treatment of pelvic floor disorders such as sacral neuromodulation therapy. In one example, this present disclosure relates to an implantable medical electrical lead having a stimulation electrode adapted to be implanted in a sacral foramen near the sacral nerve to stimulate of a bundle of sacral nerve fibers and adapted to provide chronic stability of the stimulation electrode in the sacral foramen. Moreover, the present disclosure relates to the method of implantation and anchoring of the medical electrical lead electrodes in operative relation to a selected body tissue, such as the sacral nerve to allow for stimulation within the sacral foramen.

Implantable electrical stimulation systems are therapeutic in a variety of diseases and disorders. For example, spinal cord stimulation systems have been used as a therapeutic modality for treatment of chronic pain syndromes. Deep brain stimulation has been useful for treating refractory chronic pain syndromes, movement disorders, and epilepsy. Peripheral nerve stimulation has been used to treat chronic pain syndrome and pelvic floor disorders. Other applications are under investigation.

Pelvic floor disorders such as urinary incontinence, urinary urge/frequency, urinary retention, pelvic pain, bowel dysfunction (constipation, diarrhea), and sexual dysfunction are bodily functions influenced by the sacral nerves. Specifically, urinary incontinence is the involuntary control over the bladder that is exhibited in various patients. Incontinence is primarily treated through pharmaceuticals and surgery. Pharmaceuticals may not adequately resolve the issue and can cause unwanted side effects, and a number of the surgical procedures have a low success rate and are not reversible. Several other methods have been used to control bladder incontinence, for example, vesicostomy or an artificial sphincter implanted around the urethra. These solutions also have drawbacks. In addition, some disease states do not have adequate medical treatments.

The organs involved in bladder, bowel, and sexual function receive much of their control via the second, third, and fourth sacral nerves, commonly referred to as S2, S3 and S4 respectively. Electrical stimulation of these various nerves has been found to offer some control over these functions. Several techniques of electrical stimulation may be used, including stimulation of nerve bundles within the sacrum. The sacrum, generally, is a large, triangular bone situated at the lower part of the vertebral column, and at the upper and back part of the pelvic cavity. The spinal canal runs throughout the greater part of the sacrum. The sacrum is perforated by the anterior and posterior sacral foramina that the sacral nerves pass through.

Neurostimulation leads have been implanted on a temporary or permanent basis having a stimulation electrode (at least one stimulation electrode) positioned on and near the sacral nerves to provide partial control for bladder incontinence. Temporary sacral nerve stimulation is accomplished through implantation of a temporary neurostimulation lead extending through the skin and connected with a temporary external pulse generator. A permanent neurostimulator is implanted if stimulation is efficacious and it is possible to do so in the particular patient. Permanent implantation is accomplished by implanting a permanent neurostimulation lead, extending the proximal portion of the lead body subcutaneously, and connecting its proximal end with an implantable pulse generator, or IPG, implanted subcutaneously.

In one example, a lead bearing a distal stimulation electrode is percutaneously implanted through the dorsum and the sacral foramen of the sacral segment S3 for purposes of selectively stimulating the S3 sacral nerve. The lead is advanced through the lumen of a hollow spinal needle extended through the foramen, and the single distal tip electrode is positioned adjoining the selected sacral nerve. Stimulation energy is applied through the lead to the electrode to test the nerve response. The electrode is moved back and forth to locate the most efficacious location, and the lead is then secured by suturing the lead body to subcutaneous tissue posterior to the sacrum and attached to the output of a neurostimulator IPG. Despite the suture fixation, sacral nerve stimulation leads having a single discrete tip electrode can be dislodged from the most efficacious location due to stresses placed on the lead by an ambulatory patient. Surgical intervention can then be applied to reposition the electrode and affix the lead.

The current lead designs used for permanent implantation to provide sacral nerve stimulation through a foramen have several, e.g., four, of ring-shaped, stimulation electrodes spaced along a distal segment of the lead body adapted to be passed into or through the foramen along a selected sacral nerve. Each distal stimulation electrode is electrically coupled to the distal end of a lead conductor within the elongated lead body that extends proximally through the lead body. The proximal ends of the separately insulated lead conductors are each coupled to a ring-shaped connector element in a proximal connector element array along a proximal segment of the lead body that is adapted to be coupled with the implantable neurostimulation pulse generator, or neurostimulator IPG.

The electrode array is moved back and forth with respect to the sacral nerve while the response to stimulation pulses applied through one or more of the electrodes is determined. The IPG is programmed to deliver stimulation pulse energy to the electrode providing the optimal nerve response, and the selection of the electrodes can be changed if efficacy using a selected electrode fades over time due to dislodgement or other causes.

Electrical stimulation pulses generated by the neurostimulator IPG are applied to the sacral nerve through the selected one or more of the stimulation electrodes in either a unipolar or bipolar stimulation mode. In one unipolar stimulation mode, the stimulation pulses are delivered between a selected active one of the stimulation electrodes and the electrically conductive, exposed surface of the neurostimulator IPG housing or can that provides a remote, indifferent, or return electrode. In this case, efficacy of stimulation between each stimulation electrode and the neurostimulator IPG can electrode is tested, and the most efficacious combination is selected for use. In a further unipolar stimulation mode, two or more of the stimulation electrodes are electrically coupled together providing stimulation between the coupled together stimulation electrodes and the return electrode.

In a bipolar stimulation mode, one of the distal stimulation electrodes is selected as the indifferent or return electrode. Localized electrical stimulation of the sacral nerve is effected between the active stimulation electrode or electrodes and the indifferent stimulation electrode.

A issue associated with implantation of permanent and temporary neurostimulation leads involves placing and maintaining the discrete ring-shaped electrode or electrodes in casual contact, that is in location where slight contact of the electrode with the sacral nerve may occur or in close proximity to the sacral nerve to provide adequate stimulation of the sacral nerve, while allowing for some axial movement of the lead body.

In some examples, physicians spend a great deal of time with the patient under a general anesthetic placing the leads due to making an incision exposing the foramen and due to the difficulty in optimally positioning the small size stimulation electrodes relative to the sacral nerve. In other examples, an incision is made in the skin and a needle and guide are placed into the foramen. The patient is exposed to dangers associated with extended periods of time under a general anesthetic in order to get adequate placement. Movement of the lead, whether over time from suture release or during implantation during suture sleeve installation, is to be avoided. Also, unintended movement of any object positioned proximate a nerve may cause unintended nerve damage. Moreover, reliable stimulation of a nerve entails consistent nerve response to the electrical stimulation that, in turn, entails consistent presence of the stimulation electrode proximate the sacral nerve. But, too close or tight a contact of the electrode with the sacral nerve can also cause inflammation or injury to the nerve diminishing efficacy and possibly causing patient discomfort.

Once the optimal electrode position is attained, the lead body is fixed to retard lead migration and dislodgement of the electrodes from the optimal position employing sutures or sacral lead fixation mechanisms. However, it is desirable to avoid use of complex fixation mechanisms.

Once fixation is completed, the proximal lead body is typically bent at about 90° and tunneled subcutaneously to a remote site where its proximal connector elements are coupled to the neurostimulator IPG which is then implanted at the remote site. In this process some axial and lateral dislodgement of the stimulation electrodes can also occur.

It is generally desirable to minimize surgical trauma to the patient through surgical exposure of the tissue and sacrum. It is preferred to employ a minimally invasive, percutaneous approach in a path extending from the skin to the foramen that the neurostimulation lead is extended through.

One such percutaneous approach for implantation includes a temporary neurostimulation lead that extends through the patient's skin and is attached to an external pulse generator. Typically, the external pulse generator and exposed portion of the lead body are taped to the skin to inhibit axial movement of the lead body. When a stimulation time period ends, the lead is removed through the skin by application of traction to the exposed lead body, and the incision is closed. The neurostimulation lead bodies are formed with surface treatment or roughening in a portion proximal to the neurostimulation electrode expected to extend from the foramen to the patient's skin that is intended to increase the resistance to unintended axial dislodgement of the lead body to stabilize the electrode. A length of the lead body is formed with indentations or spiral ridges or treated to have a macroscopic roughening.

### SUMMARY

To summarize the current techniques of implantable neurostimulation for sacral nerve therapy, a small lead body is placed along the trajectory of a sacral nerve. Efficacy is often linked to the ability to place and maintain the lead in a proper position proximate the sacral nerve. The closer the lead is to the nerve, the more likely that stimulation of the lead will cause nerve activation and achieve therapy efficacy. Nerve trajectory can vary from patient to patient, and lead placement can be a difficult process for implanters. Difficulties for clinicians can include identification of the nerve or the foramen opening of which the nerve is located. This difficulty may preclude some clinicians from performing the implant, which can affect the number of patients who have access to such procedures.

In a first aspect, the present disclosure is directed to the disposition within a blood vessel of an implantable medical lead for neurostimulation in sacral nerve therapy. In one example, the implantable medical lead is operably coupled to a pulse generator via a direct mechanical and electrical connection or via a wireless connection in which the pulse generator transmits an electrical signal to provide stimulation pulses without a mechanical connection to the implantable medical lead. The implantable medical lead can be disposed within a vein proximate the sacrum, such as a lateral or medial sacral vein for the sacral nerve targets, or a tibial vein if the target is the tibial nerve, or a pudendal vein if the target is the pudendal nerve. In some examples, the implantable medical lead can be disposed within the saphenous vein if the target is the saphenous nerve or the tibial nerve. The implantable medical lead can be held in place with a fixation mechanism, such as a fixation mechanism that engages the vein, for example against the vein wall within the vein lumen, engages surrounding tissue. An example of a fixation strategy is a small stent-like structure which maintains the electrodes in contact with the vessel wall. During disposition, the implantable medical lead an be visualized via a technique such as fluoroscopy with a contrasting agent to determine the location of the distal end of the lead. In some examples, the distal end of the lead, such as proximate the electrode array, may include markers to aid in the visualization of the lead with respect to the position of the nerve. The implantable medical lead can be steered through the vasculature via a catheter or stylet to an appropriate location within the vessel proximate the nerve. Alternatively, the lead can be guided through the vasculature using magnetic guidance. External magnets generate a field which provides the ability to track the lead in the three-dimensional space.

In one example, the present disclosure includes an implantable medical lead for sacral neuromodulation therapy, such as the treatment of pelvic floor disorders. The implantable medical lead includes a lead body having a distal portion. An electrode array is electrically coupled to the lead body and configured to generate a stimulation field. A stent is coupled to the distal portion. The stent is configured to anchor the implantable medical lead against an interior wall of a blood vessel, such as a sacral vein, a tibial vein, or a pudendal vein, or, in some examples, a saphenous vein (great saphenous vein). For example, the stent is configured to maintain the electrode array in position with respect to the vessel. An electrode array can effect the stimulation field from within the vessel to stimulate a selected nerve. In one example, the stent can carry the electrode array to effect the stimulation field.

In another example, the present disclosure includes an implantable medical system having an implantable lead and a neurostimulator for sacral neuromodulation therapy, such as the treatment of pelvic floor disorders. The implantable medical lead includes a lead body having a distal portion. An electrode array is electrically coupled to the lead body and configured to generate a stimulation field. A fixation mechanism is coupled to the distal portion. The fixation mechanism is configured to anchor the implantable medical lead in place within a vessel so as to effect the stimulation field from within a vessel. One example of a fixation mechanism is a stent that can couple to the inside of a wall of the vessel. Another example includes thin film electrodes disposed on the fixation mechanism. The implantable lead is electrically coupled to the neurostimulator via a wireless receiver coupled to the lead body or via a wired connection to the neurostimulator. The electrodes may also be utilized to sense signals such as electrical signals from muscle or nerve tissue or signals evoked from stimulation. Another example of a fixation mechanism includes tined leads.

In another example, the present disclosure includes a method of disposing an implantable medical lead within a blood vessel, such as a sacral vein, a tibial vein, a saphenous vein, or a pudendal vein, so as to effect a stimulation field from within the blood vessel to stimulate a selected nerve, such as a sacral nerve. In this example, the implantable medical lead is disposed within the blood vessel and steered into position via a steering tool. Additionally, the implantable medical lead can include features to steer the implantable medical lead, as a steerable medical lead, and navigate the vasculature swiftly. The steering tool can include a catheter or stylet. The implantable medical lead can be steered into position under a visualization, such as fluoroscopy, using a contrasting agent within the blood stream. Once in position, a fixation mechanism can be deployed to anchor the implantable lead in place, such as to anchor an electrode array in place. One example fixation mechanism can be an expandable stent that includes a first position, or compressed position, and a second position, or expanded position. The implantable medical lead can be steered through the vasculature with the fixation mechanism in the compressed position. Once the implantable medical lead is in position, such as the implantable medical lead has been tested to determine whether a stimulation field can apply stimulation therapy to the selected nerve, the fixation mechanism can be transitioned to the deployed position. In another example, tined leads can be introduced from a puncture site of the venous system to the site employing a stiffening stylet with a tip that can be formed with a curve extended down the lead lumen to advance it through the venous system and proximate the selected nerve. Percutaneous lead introducers are used to access the puncture site. The tines fold against the introducer lumen and the vein wall after the lead distal end exits the introducer lumen.

In another aspect, the present disclosure is directed to guided implantation of the medical lead through the body to a nerve of interest for neurostimulation via magnetic and ultrasonic mechanisms for sacral neuromodulation therapy, such as the treatment of pelvic floor disorders. The magnetic and ultrasonic mechanism are adapted to track the implantable medical lead through the body to the therapy site. In one example, the lead body, such as the distal tip, may include a magnetic mechanism. The magnetic mechanism can include a magnet, electromagnetic field generator, magnetic sensor, or detectable feature that can be detected from outside the body. In another example, a stylet or introducer may include the magnetic mechanism near the distal tip. In the former example, the magnetic mechanism may be left in the patient after implantation of the medical lead and surgical tools are removed. In the latter example, the magnetic mechanism can be withdrawn from the patient after the medical lead is positioned proximate the nerve of interest. The magnetic mechanism can be detected from outside of the body and imaged in a visualization. In still another example, the lead implanting hardware, such as the introducer or the stylet, can include an ultrasonic sensor such as an imaging array disposed at the distal end of the lead implanting hardware. The ultrasonic sensor, in some examples, may provide a signal to a monitor outside of the body that can provide an indication, such as a visualization, that may distinguish between tissues of varying threshold densities or tissue classifications such as hard and soft tissue, or various soft tissues, in which such tissues can include muscle, fat, and bone of the patient.

In one example, a magnetic sensor can be disposed on a brace positioned relative to the patient and external to the patient. The magnetic sensor can be configured to detect the position, such as the position in space, of an electrode array or distal tip of an implantable medical lead. The detected position can be provided to a processing device that may combine the position of the of the implantable medical lead with a medical image of the patient and provide a visualization of the implantable medical lead travelling through the patient relative to features of the anatomy. Using a combination of the magnetic sensor and a program, the visualization may appear to overlay the implantable medical lead onto the patient's body at a location within the patient.

The present disclosure is directed to an example of a simpler sacral neuromodulation therapy delivery that is less dependent on sacral nerve trajectory, which could expand the number of clinicians able to provide the procedure and create more access to sacral nerve therapy for patients. Further, the simpler procedure can reduce time a patient may spend under anesthesia and provide for a more repeatable procedure. The medical lead of the present disclosure provides therapy to the sacral nerve in the sacral foramen. In such examples, the fixation may vary. Such examples can include tines, a coiled lead, a stent, and a spring.

### BRIEF DESCRIPTION OF THE DRAWINGS

FIG. 1 is a perspective view illustrating an example implantable medical system for use with sacral neuromodulation therapy.

FIG. 2 is an exploded perspective view illustrating an implantable medical device useful with the system of FIG. 1.

FIG. 3A is a schematic front view illustrating an example sacrum that include foramina through which sacral nerves may extend.

FIG. 3B is a schematic perspective view illustrating an example sacrum illustrating example positioning of the sacral veins.

FIG. 4 is a plan view illustrating an example implantable medical lead that can be used with the implantable medical system of FIG. 1.

FIG. 5 is a side sectioned view illustrating the sacrum of FIG. 3 having a sacral nerve extending from a foramen, and the example implantable medical lead of FIG. 4 deployed into a blood vessel and configured to provide stimulation to the sacral nerve.

FIG. 6 is a schematic view illustrating a system that can be configured to visualize the implantation of a medical lead and system of FIG. 1 proximate the sacrum of FIG. 3.

### DETAILED DESCRIPTION

Aspects of the present disclosure provide for implantable medical devices, methods of manufacturing such implantable medical devices, and implantable medical device systems including such implantable medical devices.

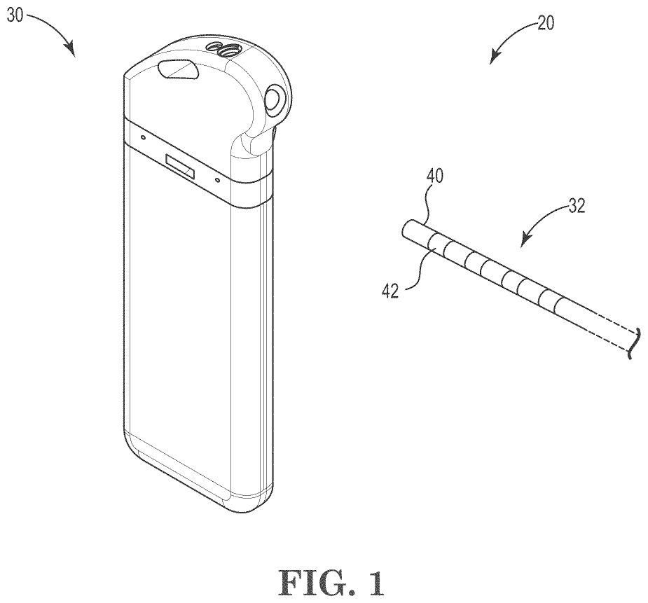

FIG. 1 illustrates an implantable medical device system 20. System 20 includes an implantable medical device (IMD) 30 and an implantable medical lead 32. In general terms, the implantable medical device 30 may be of various types, such as a device for producing electrical stimulation or for sensing physiological signals for various medical applications such as neurological or cardiac therapy. The implantable medical lead 32 includes a proximal end 40 of a lead body in which a series of electrical contacts 42 are located. Each electrical contact 42 has a corresponding conductor within the lead body that extends to a distal end (not shown) where a series of electrodes are present. During use, the proximal end 40 is inserted into the implantable medical device, establishing electrical interface between the electrical contacts 42 of the implantable medical lead 32 and electrical connectors carried by the implantable medical device 30. Stimulation signals generated by the implantable medical device 30 are delivered to the distal end of the implantable medical lead 32 and to targeted tissue and/or signals sensed by the distal end of the implantable medical lead 32 at the targeted tissue are delivered to the implantable medical device 30. The systems of the present disclosure can optionally include one or more additional components, such as one or more handset programmers configured and programmed to wirelessly interface with the implantable medical device 30.

In some examples, the system 20 and the implantable medical device 30 is configured to be useful or appropriate for providing stimulation therapy to a patient, and in particular sacral neuromodulation. In some examples, the system 20 can be described as an implantable programmable neuromodulation system that delivers electrical stimulation to the sacral nerve. Sacral neuromodulation therapy provided by the system 20 can be indicated for the management of the chronic intractable functional disorders of the pelvis and lower urinary or intestinal tract including overactive bladder, fecal incontinence, and nonobstructive urinary retention.

Sacral neuromodulation creates an electrical field near the sacral nerve to modulate the neural activity that influences the behavior of the pelvic floor, lower urinary tract, urinary and anal sphincters, and colon. The system 20 is configured to use current controlled stimulation to generate an electric field to modulate the sacral nerve. Electrical stimulation is delivered using metal electrodes provided with the implantable medical lead 32, which carry current in the form of electrons, to biological tissue, which carries current in the form of ions. An interface between the electrode and the tissue includes non-linear impedance that can be a function of the voltage across that interface. During current-controlled stimulation, an amount of current is regulated. The voltage is changed according to the actual value of impedance, such that changes in impedance will not affect the total amount of current delivered to the tissue. Current controlled waveforms can ensure that the electric field in the tissue is independent of electrode polarization or the voltage drop across the electrode-electrolyte interface. Alternatively, the systems of the present disclosure can be configured or programmed to use voltage-controlled stimulation.

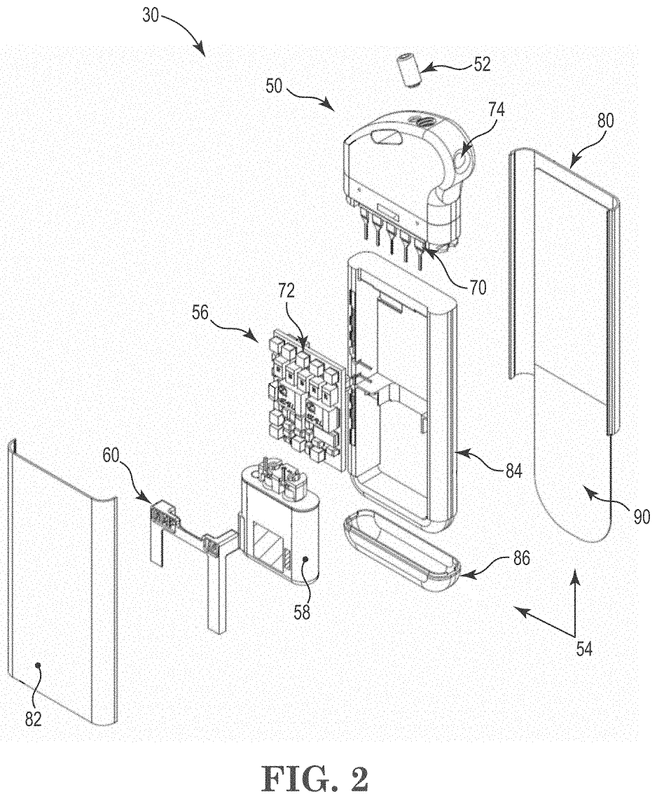

FIG. 2 illustrates an example of the implantable medical device 30 appropriate, for example, for generating the sacral neuromodulation therapy stimulation signals. The implantable medical device 30 can be configured to provide a small form factor, e.g., a volume on the order of approximately 3 cubic centimeters in some non-limiting embodiments, while generating desired stimulation signals over an extended lifetime. The implantable medical device 30 can serve as the power source of the sacral neuromodulation therapy described above in some embodiments. The smaller form factor or size as compared to conventional stimulation-type implantable medical devices can allow for a smaller implant location incision and smaller subcutaneous pocket, which may result in a more discreet implant. In some embodiments, the implantable medical device 30 incorporates various features described below that facilitate the small form factor size while providing desired performance attributes, such as remotely programmable stimulation signals or electrical pulses at therapy levels of interest, magnetic resonance imaging (MM) compatibility, and remote charging.

In some embodiments, the implantable medical device 30 includes or defines a connector enclosure assembly 50, a set screw 52, a main enclosure assembly or can 54, electrical circuitry 56, a battery 58, and an optional desiccant assembly 60. Details on the various components are provided below. In general terms, the electrical circuitry 56, the battery 58 and the desiccant assembly 60 are maintained within the can 54. The battery 58 is electrically coupled to the electrical circuitry 56. The connector enclosure assembly 50 is assembled to the can 54, and includes one or more conductor fingers 70 that are electrically connected to individual circuitry components, and in particular contact pads 72, of the electrical circuitry 56. With this construction, electrical signals generated by the electrical circuitry 56 are delivered to the connector enclosure assembly 50 via the conductor fingers 70. The connector enclosure assembly 50 further forms or defines an entryway 74 sized to receive the proximal end 40 of the implantable medical lead 32. Electrical connectors provided with the connector enclosure assembly 50 interface with the electrical contacts 42 and are electrically connected to respective ones of the conductor fingers 70, thereby connecting the electrical circuitry 56 with implantable medical lead 32. The set screw 52 provides an electrical ground between the implantable medical lead 32 as inserted into the entryway 72 and the can 54.

In an alternate example, the medical device and implantable medical lead are configured for wireless signal transmission. For example, the medical device can include a transmitter and receiver electrically coupled to a transmitter and receiver on the implantable medical lead to provide or exchange signals between the medical device and the implantable medical lead.

The can 54 can assume various forms appropriate for maintaining the electrical circuitry 56 and the battery 58, as well as for assembly with the connector enclosure assembly 50. In some embodiments, the can 54 includes opposing shield bodies 80, 82, an insulator cup 84 and an end cap 86. The shield bodies 80, 82 can be formed of a surgically safe, robust material, e.g., titanium, such as a titanium alloy 6A1-4V ELI alloy per ASTM F136, and collectively generate a sleeve, e.g., the shield bodies 80, 82 can be secured to one another by, for example, laser seam welding applied to the interfacing edges. The sleeve, in turn, defines an open volume sized and shaped to receive the insulator cup 84. To facilitate final construction, a pressure sensitive adhesive liner 90 can be provided with the first shield body 80 that is removed prior to assembly to the insulator cup 84. A bottom opening to the sleeve collectively defined by the shield bodies 80, 82 is closed by the end cap 86. The end cap 86 and the connector enclosure assembly 50 can be assembled (e.g., welded) to the shield bodies 80, 82 to provide a hermetically sealed case.

The insulator cup 84 serves as a chassis, sized and shaped to fit snugly between the shield bodies 80, 82. The insulator cup 84 spatially secures the electrical circuitry 56 and the battery 58 via appropriately sized and shaped cavities. The insulator cup 84 can be formed of an electrically non-conductive or insulative material, such as a polymer.

The electrical circuitry 56 can include various electrical components and connections appropriate for providing, in some non-limiting embodiments, a pulse generator for therapy stimulation, e.g., a constant current stimulation engine, sensing circuitry for measuring physiological parameters, telemetry for communication with external devices (e.g., inductive telemetry at 175 KHz), memory, and a recharge circuit in some non-limiting embodiments. For example, the electrical circuitry 56 can deliver stimulation signals to the contact pads 72, and can process or act upon sensed signals received at the contact pads 72. The electrical circuitry 56 optionally provides various stimulation signal parameters, for example current controlled amplitude with a resolution of 0.1 mA steps, an upper limit of 12.5 mA, and a lower limit of 0.0 mA; a rate of 3-130 kHz; pulse width increments of 10 μs steps with a maximum of 450 μs and a minimum of 20 μs.

The battery 58 can assume various forms appropriate for generating desired stimulation signals, and in some embodiments is a rechargeable battery. For example, the battery 58 can incorporate lithium ion (Li+) chemistry, although other battery constructions known in the art are also acceptable.

The desiccant assembly 60 is sized and shaped for mounting within the can 54, and provides or carries an appropriate desiccant material to promote a dry environment within the can 54.

The connector enclosure assembly 50 can be mounted to the can 54 in a hermetically sealed fashion. The conductor fingers 70 and the ground conductor 124 are arranged to extend to a corresponding one of the contact pads 72, and are welded, e.g., pressure gas welding. The desiccant assembly 60 can be placed into the can 54 following the welding process, or otherwise delayed until a remaining step is to add the second shield body 82. In this manner, the desiccant is exposed to the ambient conditions for only a short time prior to the interior of the can 54 being isolated from the exterior. This can preserve the effectiveness of the desiccant.

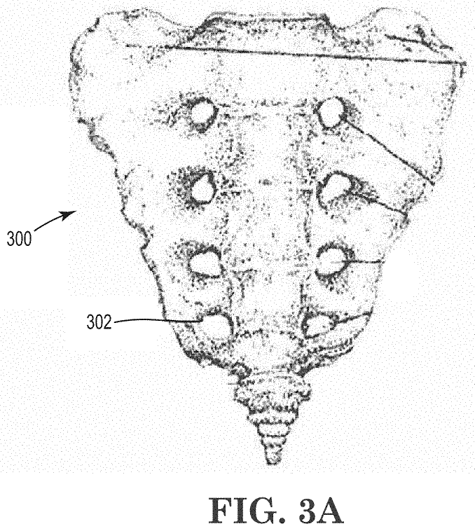

FIG. 3A illustrates an example sacrum 300. The sacrum 300 includes a plurality of foramina, such as foramen 302. The pelvic surface of the sacrum (illustrated) is concave from the top, and curved slightly from side to side (a convex dorsal surface is opposite the pelvic surface and not shown). Its middle part is crossed by four transverse ridges, which correspond to the original planes of separation between the five sacral vertebrae. The body of the first segment is large and has the form of a lumbar vertebra; the bodies of the next bones get progressively smaller, are flattened from the back, and curved to shape themselves to the sacrum, being concave in front and convex behind. At each end of the transverse ridges, are the four anterior sacral foramina, diminishing in size in line with the smaller vertebral bodies. The foramina give exit to the anterior divisions of the sacral nerves and entrance to the lateral sacral arteries. Each part at the sides of the foramina is traversed by four broad, shallow grooves, which lodge the anterior divisions of the sacral nerves.

There are five paired sacral nerves, half of them arising through the sacrum on the left side and the other half on the right side. Each nerve emerges in two divisions: one division through the anterior sacral foramina and the other division through the posterior sacral foramina. The nerves divide into branches and the branches from different nerves join with one another, some of them also joining with lumbar or coccygeal nerve branches. These anastomoses of nerves form the sacral plexus and the lumbosacral plexus. The branches of these plexus give rise to nerves that supply much of the hip, thigh, leg and foot. The sacral nerves have both afferent and efferent fibers and are responsible for part of the sensory perception and the movements of the lower extremities of the human body. From the S2, S3 and S4 arise the pudendal nerve and parasympathetic fibers whose electrical potential supply the descending colon and rectum, urinary bladder and genital organs. These pathways have both afferent and efferent fibers and, this way, they are responsible for conduction of sensory information from these pelvic organs to the central nervous system (CNS) and motor impulses from the CNS to the pelvis that control the movements of these pelvic organs.

FIG. 3B illustrates an example sacrum, such as the sacrum 300, illustrating the pelvic surface. Sacral nerves, and other nerves of interest, can be positioned alongside veins near the sacrum and legs. For example, sacral nerves of interest can be positioned near the sacrum and proximate a plurality of sacral veins including left lateral sacral vein 304, or LSV, right lateral sacral vein 306, or RSV, and median sacral vein 308, or MSV. The lateral sacral veins LSV and RSV, 304, 306 accompany the lateral sacral arteries on the anterior surface of the sacrum and end in the hypogastric vein. The median sacral vein MSV 308 (or middle sacral veins) accompanies the corresponding artery along the front of the sacrum, and joins to form a single vein, which ends in the left common iliac vein; sometimes in the angle of junction of the two iliac veins. Additionally, nerves of interest for the treatment of pelvic floor disorders can be located proximate the sacrum and near the tibial vein and pudendal vein or near the saphenous vein. For example, nerves of interest for the treatment of pelvic floor disorders can be located proximate the sacrum and near the tibial vein and pudendal vein or near the saphenous vein.

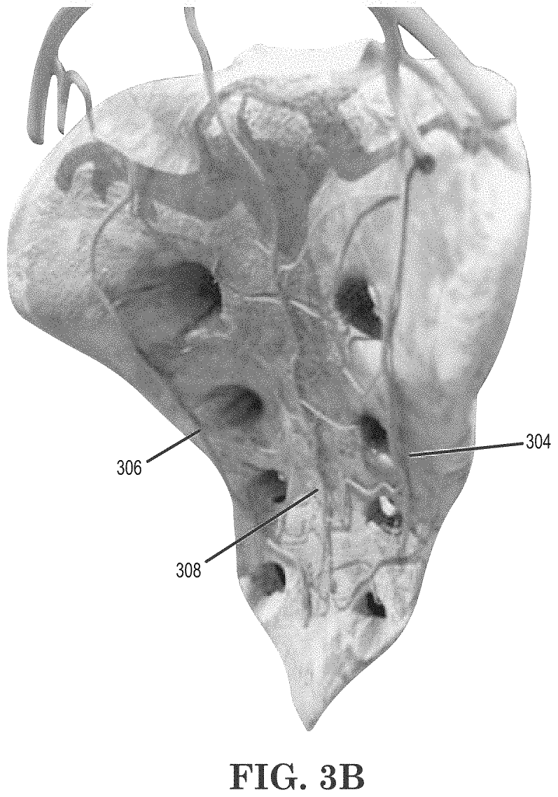

FIG. 4 illustrates a schematic example of an implantable medical lead 110, which can be an example of implantable medical lead 32, that may be applied to provide sacral nerve stimulation that allow for non-direct contact stimulation of the sacral nerves and comprises a lead body 115, an electrode array 120 configured to provide or receive electrical signals such as stimulation signals to the sacral nerve, and, in some examples, a fixation mechanism 122 to hold the electrode array 120 in place. In one example of an implantable medical lead 110 with a fixation mechanism 122, the fixation mechanism 122 is configured to prevent or reduce the likelihood of migration of the electrode array 120.

The electrode array 120 can include one or more electrodes, such as four electrode 125, 130, 135, and 140. In the illustrated example, the electrode array 120 is disposed on a lead distal end 145. In another example, an implantable medical lead could include an electrode array with a single electrode disposed on the distal end of the lead body. Other configurations are contemplated. The electrode array 120 can be configured to provide a steerable stimulation field—for example, a lobe of the field can be movable with respect to the lead distal end 145—to allow the field to be created or adjusted in the direction of the nerve once the placement of the lead is complete. An outer diameter of the lead body 115 can be in the range of about 0.5 mm to about 2 mm. In one example, the electrodes 125, 130, 135 and 140 are made of a solid surface, bio-compatible material such as a pad or tube formed of platinum, platinum-iridium alloy, or stainless steel, of about 3.0 mm in length that does not degrade when electrical stimulation is delivered through. The electrode can further be separated by insulator bands.

Each stimulation electrode in the electrode array 120, such as 125, 130, 135, and 140, is electrically coupled to the distal end of a coiled wire lead conductor within the elongated lead body 115 that extends proximally through a distal portion 150 and through a proximal portion 155 of the lead body 115. The proximal ends of the separately insulated lead conductors can each be coupled to respective ring-shaped connector elements 165, 170, 175, and 180 in a proximal connector element array 160 along the proximal portion 155 of the lead body 115 adjacent the lead proximal end 185. The conductor wires can be formed of an MP35N alloy and are insulated from one another within an insulating polymer sheath such as polyurethane, fluoropolymer, or silicone rubber. An example diameter of the lead body 115 is 1.3 mm but smaller diameters are also contemplated. The lead conductor wires are separately insulated by an insulation coating and are wound in a quadra-filar manner having a common winding diameter within the outer sheath. The coil formed by the coiled wire conductors defines a lead body lumen of the lead body 115. In some examples, a further inner tubular sheath could be interposed within the aligned wire coils to provide the lead body lumen.

The connector elements 165, 170, 175, and 180 are adapted to be coupled with a neurostimulator IPG, such as implantable medical device 30, additional intermediate wiring, or other stimulation device adapted to be implanted subcutaneously. An example of such an implantable pulse generator is available under the trade designation Medtronic InterStim Neurostimulator from Medtronic, Inc. Electrical stimulation pulses generated by the implantable medical device 30 are applied to the sacral nerve through one or more of the stimulation electrodes 125, 130, 135 and 140 in a unipolar or bipolar stimulation mode.

The axial lead body lumen (not shown) can extend the length of the lead body 115 between a lumen proximal end opening at lead proximal end 185 and a lumen distal end opening at lead distal end 145. A guide wire or stiffening stylet 200 having a handle 205 and a straight wire 210 attached to the handle 205 can be inserted through the lead body lumen to assist in implantation of the implantable medical lead 110. The straight wire 210 or stylet wire can be made of a solid wire such as tungsten or stainless steel. In some examples, the fixation mechanism 122 can be covered with an introducer lumen to assist in implantation. Once the implantable medical lead 100 is properly positioned within the body, the introducer lumen can be removed to expose the fixation mechanism 122, and the guide wire 210 is also removed.

In implanting the medical lead 110, an introducer can be placed over the lead distal portion 150 to cover the electrode array 120 and the fixation mechanism 122, and the stylet 200 is disposed within the lead body 115 so that its distal tip closes a lumen distal opening. The assembly is advanced percutaneously at a selected angle until, in one example, the assembly recaches a selected foramen. A needle for electrical stimulation via a pulse generator can be included in the assembly to apply electrical stimulation to locate a nerve of interest. The assembly is distally advanced through the foramen, such as through the subcutaneous tissue to the desired position. The stylet 200 and the introducer are retracted from the patient to expose or deploy the fixation mechanism 122 and leave the medical lead 110 in place.

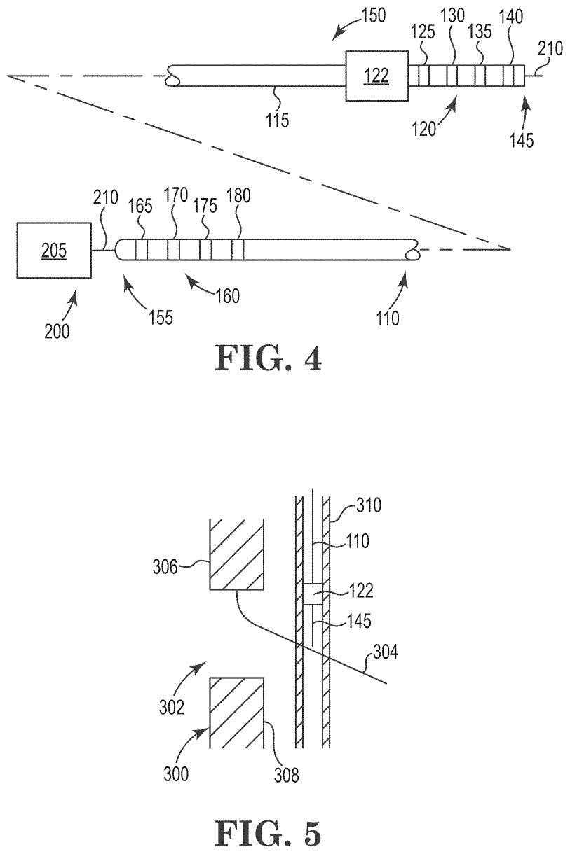

FIG. 5 illustrates a schematic view of the implantable medical lead 110 deployed proximate a sacrum, such as sacrum 300. The illustration indicates a side view of the sectioned sacrum, which includes a foramen, such as foramen 302. An example sacral nerve 304 extends from the sacrum 300 through the foramen 302. The example sacrum 300 can include a first surface 306 and an opposite second surface 308, which may correspond with one of the pelvic and dorsal or dorsal and pelvic surfaces, respectively. A blood vessel 310, such as a sacral vein, can be used to provide sacral neuromodulation, so as to effect a stimulation field from within the blood vessel 310 to stimulate a selected nerve, such as a sacral nerve 304. In this example, the implantable medical lead 110 is disposed within the blood vessel 310 and steered into position via a steering tool. The steering tool can include a catheter or stylet. The implantable medical lead 110 can be steered into position under a visualization, such as fluoroscopy, using a contrasting agent within the blood stream. Once in position, a fixation mechanism 122 can be deployed to anchor the implantable lead in place, such as to anchor an electrode array in place.

In one example, the fixation mechanism 122 is deployed lumen of the blood vessel 310 engage the walls of the blood vessel 310 in a manner to still permit the flow of blood and to maintain the position of the electrode array 120, which provides stimulation to the sacral nerve 304 within the blood vessel 310. In one example, all of the electrodes configured to engage the sacral nerve 304 with non-direct stimulation are disposed and maintained within the blood vessel 310

In one example, the implantable medical lead 110 can be inserted into the foramen 302 in a first state, such as an undeployed state. For instance, the fixation mechanism 122 can be compressed in a first state. Once in the steered into position within the blood vessel 310, the implantable medical lead 110 can be transitioned to a second state, such as a deployed state. For instance, the electrode array 120 can be positioned to provide or effect the stimulation field of the sacral nerve from within the blood vessel 310, and, after testing, the fixation mechanism 122 can be expanded. In the deployed state, the fixation mechanism 122 can maintain the stimulation field to effect stimulation of the sacral nerve 304.

In one example, the fixation mechanisms are stents. Stents are generally elongated devices having an oval or circular cross-section that are radially expandable to radially urge against the walls of a blood vessel 310 or another anatomical lumen, after implantation into the body lumen. In general, the stents may be an expandable or a self-expanding stent. Expandable stents generally are conveyed to the area to be treated on an expandable device. For insertion into the body, the stent is positioned in a compressed configuration on the delivery device. For example, the stent may be crimped onto a balloon that is folded or otherwise wrapped about the distal portion of a body that is part of the delivery device. After the stent is positioned within the foramen 302, it is expanded by the delivery device, causing the diameter of the stent to expand. For a self-expanding stent, commonly a sheath is retracted, allowing the stent to expand. An example stent can comprise a plurality of elongated strut portions and a plurality of flexible crown portions extending from the strut portions. The strut portions may intersect at vertices. When the stent is radially expanded the flexible crown portions assume a diameter recoil prevention position. Metallic stents can comprise a variety of biocompatible metals including stainless steel, titanium, gold, nickel/titanium alloys, such as nitinol, platinum, and platinum-tungsten alloys. These metallic materials are sufficiently flexible to allow the stent to be compressed and expanded, but also provide sufficient radial strength to maintain the stent in the expanded configuration and apply adequate force to the walls of the foramen to hold the stent in place. Polymeric stents may be constructed from biostable polymers appropriate for the stents that can include polyethylene, polypropylene, polymethyl methacrylate, polyesters, polyamides, polyurethanes, polytetrafluoroethylene (PTFE), polyvinyl alcohol, and other suitable polymers. These polymers may be used alone or in various combinations to give the stent unique properties such as to form biostable stents with a biodegradable or bioerodable coating that may reduce inflammation, control tissue ingrowth, and additionally, release a drug.

In one example the fixation mechanism 122 is a cylindrical stent having a generally cylindrical body. The cylindrical body includes an elongate side that is configured to extend along an axis of the lumen and a generally circular or oval cross-section that is configured to radially expand to engage the walls of the lumen in a deployed or expanded state. In another the example, fixation mechanism 122 is a stent having a generally hour-glass shaped body. The body includes an elongate side that is configured to extend along an axis of the lumen and a generally circular or oval cross-section that is in which both ends of the body includes a diameter of the circular cross-section that is generally larger than the size or diameter of the middle portion of the body. The body is configured to radially expand to engage the blood vessel at the walls of the lumen in a deployed or expanded state.

Electrodes for an electrode array can be disposed on the fixation mechanism 122 For example, in an implantable medical lead 110 that includes a fixation mechanism configured as a stent or similar device, an electrode array can comprise a ring electrode disposed around the body of the stent or similar device. For instance, the electrode array can include a plurality of spaced-apart ring electrodes along the elongate side or length of the electrode body, or other electrodes. Additionally, a ring can carry a plurality of electrodes spaced apart and disposed around the ring. In another example, a fixation mechanism configured as a stent or similar device and can include an electrode array having a plurality of pad electrodes disposed on the body of the stent or similar device, such as on the joints of the intersecting struts. The electrode array can extend along the length of the body, or a portion of the length of the body, and along an arc of the circumference of the body or along the entire circumference of the body. Implantable lead can include a fixation mechanism configured as a stent or similar device and can include an electrode array having a plurality of pad electrodes disposed on the ends of the body. The electrode array can extend along the length an arc of the circumference of the end or along the entire circumference of the end. In one example, the number of electrodes in the electrode array can include more than four electrodes. For instance, the more than one electrode may correspond with a single stimulation signal. When deployed, the closest electrode corresponding with the single stimulation signal may be activated, and the remaining electrode or electrodes that also correspond with the stimulation signal may be rendered dormant.

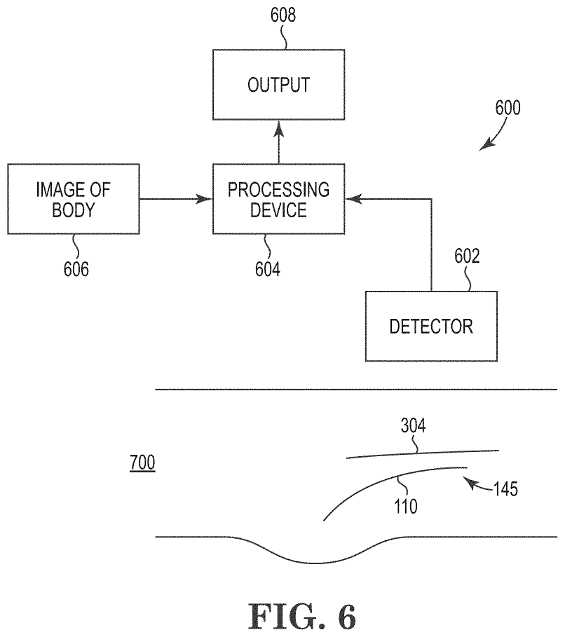

FIG. 6 illustrates a schematic view of a system 600 for guided implantation of the medical lead 110 through the body 700 to a nerve of interest for neurostimulation. For example, the system 600 can track the medical lead 110 through the body 700 such as into tissue, which may include muscle and fat and through a foramen, such as foramen 302, or such as a through a blood vessel, to a therapy site. The system 600 can include a detector 602 disposed outside of the body 700 to detect a mechanism on the implantable medical lead when disposed within the body. In one example, the detector 602 includes a sensor. The sensor is configured to remotely interface with the implantable medical lead 110, such as without mechanical contact with the detector 602 and the medical lead 110. The detector 602 generate an electrical signal that is representative of the position of the medical lead 110 within the body which is operably provided to the processing device 604. For example, the detector 602 can include a sensor operably coupled to a wire and a conductive electrical coupling that is coupled to the processing device, or the detector 602 may be wirelessly coupled to provide the electrical signal to the processing device 604. The processing device 604 may receive the electrical signal from the detector 602 and apply the electrical signal, such as parameters of the electrical signal, to determine the relative position of the medical lead within the body such as via magnetic or ultrasound techniques. In one example, the processing device can generate images of the body 700, which may be provided from another sensor or the detector 602, and apply the images of the body to the relative position of the medical lead 110 to provide a visualization of the medical lead 110 within the body 700.

For example, the mechanism may include a magnetic mechanism near the distal end 145, and may include a mechanism coupled to the implantable medical lead or the introducing hardware, e.g., stylet or introducer, included specifically for interfacing with detector, or a portion on the implantable medical lead 110 that can establish a magnetic field that is detectable by the sensor. The magnetic signal can be applied to generate an electrical signal with the sensor. The detector 602 is operably coupled to a processing device 604 to receive the signal from the sensor. The signal can be processed to determine or be indicative of a position of the mechanism in space, such as a relative position of the mechanism within the body 700 based on a strength of signal in an interface between the detector 602 and the mechanism. The processing device 604 can be configured to receive an image 606 of the body 700, including a portion of the body with the distal tip 145, which can be provided in one example also by the detector 602 or via another imaging device or prior image of the body 700. In the example, the processing device 604 can combine the image 606 with a signal received from the detector 602 and provide a visualization on an output device 608, such as a monitor or speaker operably coupled to the processing device 604.

In another example, the sensor of the detector 602 is included as an ultrasonic transducer that can provide an ultrasonic signal into the body and to the mechanism and then receive a return signal with the sensor. The detector 602 is operably coupled to a processing device 604 to receive the signal from the sensor. The signal can be processed to determine or be indicative of a position of the mechanism in space, such as a relative position of the mechanism within the body 700. The processing device 604 can be configured to receive an image 606 of the body 700, including a portion of the body with the distal tip 145, which can be provided in one example also by the detector 602 or via another imaging device or prior image of the body 700. In the example, the processing device 604 can combine the image 606 with a signal received from the detector 602 and provide a visualization on an output device 608, such as a monitor or speaker operably coupled to the processing device 604.

The processing device 604 can be configured to operate a program, such as a computer or processor readable medium storing computer or processor readable to control the processing device 604 to image the distal end 145 of the implantable medical lead 110. The processing device 604 can be a stand-alone device or configured as part of a system to store instructions and data that are executable by a processor. The processor 404 may include two or more processing cores on a chip or two or more processor chips. In some examples, the processing device 604 can also have additional processing or specialized processors (not shown), such as a graphics processor for general-purpose computing on graphics processor units, to perform processing functions offloaded from the processor. The memory may be arranged in a hierarchy and may include one or more levels of cache. Depending on the configuration and type of processing device, memory may be volatile (such as random access memory (RAM)), non-volatile (such as read only memory (ROM), flash memory, etc.), or some combination of the two. Memory can include memory devices and storage devices for storage of readable instructions, data structures, program modules or other data that can be accessed by the processing device 604. Accordingly, a propagating signal by itself does not qualify as storage media. In one example, the processing device 604 can include communication connections that can facilitate the processing device 604 to be used as part of a computer network, which is a collection of computing or processing devices and possibly other devices interconnected by communications channels that facilitate communications and allows sharing of resources and information among interconnected devices. Examples of computer networks include a local area network, a wide area network, the interne, or other network. Examples of a processing device can include medical devices that may be configured specifically for imaging of the medical lead 110 through the body 700 and computing devices that may be configured via software for imaging of the medical lead 110 through the body 700. Visualizations can be provided via a monitor or display operably coupled to the processing device 604. In one example, the display is integrated with the stand-alone, specialized medical device of the processing device 604.

Although the present disclosure has been described with reference to preferred embodiments, workers skilled in the art will recognize that changes can be made in form and detail without departing from the spirit and scope of the present disclosure.

What is claimed is:

1. An implantable medical system for treatment of a pelvic floor disorder, the system comprising:
an implantable medical device having electrical circuitry configured to perform electrical stimulation; and
an implantable medical lead operably coupled to the implantable medical device, the implantable medical lead comprising:
a lead body having a distal portion;
an electrode array electrically coupled to the lead body and configured to generate a stimulation field; and
a fixation mechanism coupled to the distal portion, the fixation mechanism configured to anchor the implantable medical lead within a blood vessel and the electrode array to effect the stimulation field on a nerve of interest for treatment of a pelvic floor disorder.

2. The system of claim 1 wherein the fixation mechanism is configured to anchor the implantable medical lead against the blood vessel.

3. The system of claim 1 wherein the fixation the blood vessel is one of a sacral vein, a tibial vein, and a pudendal vein and the electrode array effects the stimulation field on a sacral nerve.

4. The system of claim including a neurostimulator operably coupled to the implantable medical lead.

5. The system of claim 4 wherein the electrode array is electrically coupled to a wireless receiver in electrical communication with the neurostimulator.

6. The system of claim 4 wherein the implantable medical lead includes a proximal end having a proximal array electrically coupled to the electrode array and mechanically and electrically coupled to the neurostimulator.

7. The system of claim 4 wherein the neurostimulator is an implantable medical device.

8. The system of claim 1 wherein the electrode array includes four electrodes.

9. An implantable medical lead for an implantable electrical stimulation system having an implantable medical device having electrical circuitry configured to perform electrical stimulation for sacral neuromodulation therapy, the implantable medical lead comprising:
a lead body having a distal portion;
an electrode array electrically coupled to the lead body and configured to generate a stimulation field for a sacral nerve; and
a fixation mechanism coupled to the distal portion, the fixation mechanism configured to anchor the implantable medical lead to a blood vessel and effect the stimulation field.

10. The lead of claim 9 wherein the fixation mechanism includes an expandable device configurable in a delivery position and a deployed position.

11. The lead of claim 15 wherein the expandable member includes a stent.

12. A method of applying a stimulation field to a sacral nerve, the method comprising:
deploying an implantable medical lead having a lead body and an electrode array, the electrode array coupled to lead body and configured to generate the stimulation field, into a body vasculature;
steering the electrode array through the vasculature to a position in which the stimulation field can stimulate a selected sacral nerve; and
anchoring the electrode array in place within the vasculature.

13. The method of claim 12 including anchoring the electrode array in place within a sacral vein, a tibial vein, or a pudendal vein.

14. The method of claim 12 wherein the anchoring includes deploying a fixation mechanism to anchor the electrode array in place.

15. A system for visualizing an implantable medical lead within a body proximate a sacral nerve, the system comprising:
a processing device; and
a detector operably coupled to the processing device, the detector including a sensor configured to remotely interface with the implantable medical lead within the body, the sensor configured to generate an electrical signal from the interface with the implantable medical lead;
wherein the processing device is configured to determine from the electrical signal a relative position of the implantable medical lead within the body proximate the sacral nerve.

16. The system of claim 15 wherein the processing device is configured to generate a visualization of the relative position of the implantable medical lead within the body.

17. The system of claim 16 wherein the processing device includes an integrated monitor to provide the visualization.

18. The system of claim 15 wherein the sensor is a magnetic sensor or an included in an ultrasonic transducer.

19. A method for guiding implantation of a medical lead within a body proximate a sacral nerve, the method comprising:
implanting the medical lead through the body proximate the sacral nerve;
introducing a detector remote from the medical lead, the detector including a sensor to generate a signal from interfacing with the medical lead; and
determining from the electrical signal a relative position of the implantable medical lead within the body.

20. The method of claim 19 including generating a visualization a visualization of the relative position of the implantable medical lead within the body.
